SEQUENCE LISTING (2 sequences)

SEQ ID NO: 1 - DNA, 1302 bp, Homo sapiens
atggaggtggtggacccgcagcagctgggcatgttcacggagggcgagctgatgtcggtgggtatggacacgttcatccaccgcatcgactccaccgaggtcatctaccagccgcgccgcaagcgggccaagctcatcggcaagtacctgatgggggacctgctgggggaaggctcttacggcaaggtgaaggaggtgctggactcggagacgctgtgcaggagggccgtcaagatcctcaagaagaagaagttgcgaaggatccccaacggggaggccaacgtgaagaaggaaattcaactactgaggaggttacggcacaaaaatgtcatccagctggtggatgtgttatacaacgaagagaagcagaaaatgtatatggtgatggagtactgcgtgtgtggcatgcaggaaatgctggacagcgtgccggagaagcgtttcccagtgtgccaggcccacgggtacttctgtcagctgattgacggcctggagtacctgcatagccagggcattgtgcacaaggacatcaagccggggaacctgctgctcaccaccggtggcaccctcaaaatctccgacctgggcgtggccgaggcactgcacccgttcgcggcggacgacacctgccggaccagccagggctccccggctttccagccgcccgagattgccaacggcctggacaccttctccggcttcaaggtggacatctggtcggctggggtcaccctctacaacatcaccacgggtctgtaccccttcgaaggggacaacatctacaagttgtttgagaacatcgggaaggggagctacgccatcccgggcgactgtggccccccgctctctgacctgctgaaagggatgcttgagtacgaaccggccaagaggttctccatccggcagatccggcagcacagctggttccggaagaaacatcctccggctgaagcaccagtgcccatcccaccgagcccagacaccaaggaccggtggcgcagcatgactgtggtgccgtacttggaggacctgcacggcgcggacgaggacgaggacctcttcgacatcgaggatgacatcatctacactcaggacttcacggtgcccggacaggtcccagaagaggaggccagtcacaatggacagcgccggggcctccccaaggccgtgtgtatgaacggcacagaggcggcgcagctgagcaccaaatccagggcggagggccgggcccccaaccctgcccgcaaggcctgctccgccagcagcaagatccgccggctgtcggcctgcaagcagcagtga

SEQ ID NO: 2 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

(19) United States
(12) Patent Application Publication
(10) Pub. No.: US 2014/0314747 A1
(43) Pub. Date: Oct. 23, 2014

(54) PREDICTING RESPONSE TO EGFR INHIBITORS

(71) Applicant: Genentech, Inc., South San Francisco, CA (US)

(72) Inventors: Geraldine Strasser, San Francisco, CA (US); Bryan Lo, San Mateo, CA (US)

(73) Assignee: Genentech, Inc., South San Francisco, CA (US)

(21) Appl. No.: 14/206,616

(22) Filed: Mar. 12, 2014

Related U.S. Application Data
(60) Provisional application No. 61/781,187, filed on Mar. 14, 2013.

(51) Int. Cl. G01N 33/574

(52) U.S. Cl. CPC G01N 33/57492
USPC 424/133.1; 435/7.9; 424/174.1; 514/234.5

(57) ABSTRACT

Methods of predicting whether a cancer will respond to an EGFR inhibitor are provided.

### FIELD

The present invention generally relates to genetic variations associated with responsiveness to EGFR inhibitors.

### BACKGROUND

The Serine/Threonine kinase LKB1 was first identified as the gene mutated in the familial cancer syndrome Peutz-Jeghers syndrome (Hemminki et al., 1998, Nature, 391: 184-187). Subsequently, somatic mutations in LKB1 were found in a variety of sporadic cancers, including up to 30% of non-small cell lung cancers (NSCLC) (Sanchez-Cespedes, 2007, Oncogene, 26: 7825-7832). The molecular function of LKB1 as a tumor suppressor remains unknown.

Similarly, mutations in the Epidermal Growth Factor Receptor (EGFR) have long been associated with many types of cancer, including lung (Pastorino et al., 1993, J. Cell. Biochem. Suppl., 17F: 237-248). A number of pharmaceuticals targeting EGFR have been developed and used successfully in the clinic to treat several types of cancer, including erlotinib (Tarceva®, approved in 2004), gefitinib (Iressa®, 2003), panitumumab (Vectibix®, 2006) and cetuximab (Erbitux®, 2004). However, secondary resistance to these treatments frequently results (Wei et al., 2011, Anticancer Drugs, 22: 963-970; Brugger and Thomas, 2012, Lung Cancer, 77: 2-8). While activating mutations of EGFR (EGFR-Mut+) are thought to be predictive for responsiveness to EGFR targeted therapies, they are not sufficient. Furthermore, responsiveness to EGFR-targeted therapies in the absence of EGFR mutations has been observed (Piperdi and Perez-Soler, 2012, Drugs, 72 Suppl. 1: 11-19). These results suggest the need for diagnostics to better predict patient response to EGFR-targeted therapies.

Activating EGFR mutations and loss of function LKB1 mutations, while both frequently observed in lung and other types of cancer, have been reported by several groups to be mutually exclusive; that is, if one mutation is present the other is statistically less likely to occur than if the mutations were assorting randomly within the patient population (Reungwetwattana et al., 2012, Clin. Lung Cancer, 13: 252-266; Chitale et al., 2008, Oncogene, 28: 2773-2783; Koivunen et al., 2008, Br. J. Cancer, 455: 245-252; Ding et al., 2008, Nature, 455: 1069-1075). This suggests that they may be involved in the same molecular pathway, and that mutation of one or the other is sufficient to deregulate this pathway and drive tumor progression.

### SUMMARY

In some embodiments, methods for predicting whether a cancer will respond to an EGFR inhibitor are provided. In some embodiments, a method comprises determining whether the cancer comprises a LKB1 mutation, wherein the presence of the LKB1 mutation indicates that the cancer will respond to the EGFR inhibitor.

In some embodiments, methods of identifying cancer patients who are likely to benefit from an EGFR inhibitor are provided. In some embodiments, a method comprises determining whether the patient's cancer comprises a LKB1 mutation, wherein the presence of the LKB1 mutation indicates that the cancer patient will likely benefit from the EGFR inhibitor.

In some embodiments, methods of selecting a therapy for cancer patients are provided. In some embodiments, a method comprises (a) determining whether the patient's cancer comprises a LKB1 mutation; and (b) if the patient's cancer comprises a LKB1 mutation, selecting an EGFR inhibitor for the therapy.

In some embodiments, methods of treating a cancer in a mammal are provided. In some embodiments, a method comprises (a) determining whether the cancer comprises a LKB1 mutation; and (b) if the cancer comprises a LKB1 mutation, administering to the mammal a therapeutically effective amount of an EGFR inhibitor.

In some embodiments, methods of treating cancer comprising a LKB1 mutation in a mammal are provided. In some embodiments, a method comprises administering to the mammal having the cancer a therapeutically effective amount of an EGFR inhibitor. In some embodiments, prior to administering the EGFR inhibitor, the cancer was determined to comprise a LKB1 mutation.

In any of the embodiments described herein, the cancer may be a solid tumor. In any of the embodiments described herein, the cancer may be selected from a large cell carcinoma, carcinoid cancer, cancer of neuroendrocrine origin, head and neck squamous cell carcinoma (HNSCC), colorectal cancer, cervical cancer, melanoma, skin cancer, leiomyoma, gastric cancer, glioblastoma, ovarian cancer, small cell lung cancer (SCLC), non-small cell lung cancer (NSCLC), pancreatic cancer, esophageal cancer, gastric cancer and thyroid cancer. In any of the embodiments described herein, the cancer may be selected from lung cancer, pancreatic cancer, colorectal cancer, and head and neck cancer. In any of the embodiments described herein, the cancer may be a tissue selected from the tissues in Table 2.

In some embodiments, the LKB1 mutation comprises a variation in a LKB1 polynucleotide. In some embodiments, the variation in the LKB1 polynucleotide is in the coding sequence of a LKB1 polynucleotide. In some embodiments, the variation in the LKB1 polynucleotide comprises at least one variation selected from an insertion, a deletion, an inversion, and a substitution. In some embodiments, the variation in the LKB1 polynucleotide results in a frame shift in the LKB1 coding sequence. In some embodiments, the variation in the LKB1 polynucleotide is a nucleotide variation at a nucleotide position selected from Table 1 that results in (a) significantly reduced or absent levels of LKB1 protein and/or (b) expression of a LKB1 protein with significantly reduced activity. In some embodiments, the variation in the LKB1 polynucleotide is a nucleotide change selected from Table 1.

In some embodiments, the variation in the LKB1 polynucleotide results in a variation in the LKB1 polypeptide. In some embodiments, the variation in the LKB1 polypeptide is selected from an insertion, a substitution, a deletion, and a truncation. In some embodiments, at least one variation in the LKB1 polypeptide is an amino acid variation at an amino acid position selected from Table 1 that results in (a) significantly reduced or absent levels of LKB1 protein and/or (b) expression of a LKB1 protein with significantly reduced activity. In some embodiments, at least one variation in the LKB1 polypeptide is an amino acid change selected from Table 1.

In any of the embodiments described herein, the mammal may be a human.

In any of the embodiments described herein, the EGFR inhibitor may be an antibody that binds EGFR. In some embodiments, the EGFR inhibitor is selected from cetuximab, panitumumab, DL11f, and GA201.

In any of the embodiments described herein, the EGFR inhibitor may be a small molecule. In some embodiments, the EGFR inhibitor is selected from erlotinib or gefitinib.

These and further embodiments are described in the following written description.

### BRIEF DESCRIPTION OF THE FIGURES

FIG. 1 shows growth and branching of Lkb1^{wt/wt} and Lkb1^{MG/MG} mouse embryo mesenchyme-free lung tissue explants incubated with NMPP1, EGF, NMPP1+EGF, NMPP1+EGF+erlotinib (Tarceva®), or NMPP1+FGF7+erlotinib (Tarceva®), as described in Example 2.

FIG. 2 shows growth and branching of Lkb1^{wt/wt} and Lkb1^{MG/MG} mouse embryo whole mount lung tissue explants incubated with NMPP1, EGF, NMPP1+EGF, or NMPP1+EGF+erlotinib (Tarceva®), as described in Example 2.

FIG. 3A-B show (A) EGFR levels in LKB1^{wt/wt} and LKB1^{MG/MG} lungs in the presence or absence of NMPP1, and (B) EGFR tyrosine phosphorylation in LKB1^{wt/wt} and LKB1^{MG/MG} lungs in the presence NMPP1, with or without a 10 minute incubation with EGF, as described in Example 2.

FIG. 4 shows development of the cystic phenotype in LKB1^{MG/MG} pancreatic explants incubated with NMPP1, EGF, and NMPP1+EGF, as described in Example 2.

FIG. 5 shows development of the cystic phenotype in Lkb1^{wt/wt} and Lkb1^{MG/MG} pancreatic explants incubated with EGF or EGF and NMPPI, as described in Example 2.

FIG. 6 shows development of the cystic phenotype in Lkb1^{wt/wt} and Lkb1^{MG/MG} pancreatic explants incubated with EGF and NMPPI, or with EGF, NMPPI, and erlotinib (Tarceva®), as described in Example 2.

### DETAILED DESCRIPTION OF CERTAIN EMBODIMENTS

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

Before the present compositions and methods are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include the plural unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “primer” refers to a single stranded polynucleotide that is capable of hybridizing to a nucleic acid and allowing the polymerization of a complementary nucleic acid, generally by providing a free 3′-OH group.

A “HER receptor” is a receptor protein tyrosine kinase which belongs to the HER receptor family and includes EGFR (ErbB1, HER1), HER2 (ErbB2), HER3 (ErbB3) and HER4 (ErbB4) receptors. The HER receptor will generally comprise an extracellular domain, which may bind a HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a naturally occurring (“native sequence”) HER receptor or a variant thereof. Preferably the HER receptor is a native sequence human HER receptor.

The “HER pathway” refers to the signaling network mediated by the HER receptor family.

The terms “ErbB1”, “HER1”, “epidermal growth factor receptor” and “EGFR” are used interchangeably herein and refer to EGFR as disclosed, for example, in Carpenter et al. Ann. Rev. Biochem. 56:881-914 (1987), including naturally occurring mutant forms thereof (e.g. a deletion mutant EGFR as in Ullrich et al, Nature (1984) 309:418425 and Humphrey et al. PNAS (USA) 87:4207-4211 (1990)), as well as variants thereof, such as EGFRvIII. Variants of EGFR also include deletional, substitutional and insertional variants, for example those described in Lynch et al (New England Journal of Medicine 2004, 350:2129), Paez et al (Science 2004, 304:1497), and Pao et al (PNAS 2004, 101:13306).

Herein, “EGFR extracellular domain” or “EGFR ECD” refers to a domain of EGFR that is outside of a cell, either anchored to a cell membrane, or in circulation, including fragments thereof. In some embodiments, the extracellular domain of EGFR may comprise four domains: “Domain I” (amino acid residues from about 1-158, “Domain II” (amino acid residues 159-336), “Domain III” (amino acid residues 337-470), and “Domain IV” (amino acid residues 471-645), where the boundaries are approximate, and may vary by about 1-3 amino acids.

“Phosphorylation” refers to the addition of one or more phosphate group(s) to a protein, such as an EGFR receptor, or substrate thereof.

The terms “Serine/Threonine-Kinase 11,” “STK11,” “Liver Kinase B1,” and “LKB1” are used interchangeably and refer to any native LKB1 protein, coding sequence, or gene from any vertebrate source, including mammals such as primates (e.g. humans) and rodents (e.g., mice and rats), unless otherwise indicated. The term encompasses “full-length,” unprocessed LKB1 as well as any form of LKB1 that results from natural processing. The term also encompasses naturally occurring variants of NRG, e.g., splice variants or allelic variants. The sequence of an exemplary human LKB1 protein is shown at Swiss-Prot Accession No. Q15831.1. The sequence of an exemplary human LKB1 coding sequence is shown at GenBank Accession No. NM_{—}000455.4. Exemplary genetic information, including the gene location, sequence, and intron/exon boundaries, is shown at NCBI Gene ID 6794. A nonlimiting exemplary LKB1 coding sequence is shown in SEQ ID NO: 1. A nonlimiting exemplary LKB1 amino acid sequence is shown in SEQ ID NO: 2.

The term “LKB1 polynucleotide” or “nucleic acid encoding LKB1” refers to a gene or coding sequence (e.g., an mRNA or cDNA coding sequence) that encodes LKB1, unless otherwise indicated.

The term “nucleotide variation” refers to a change in a nucleotide sequence (e.g., an insertion, deletion, inversion, or substitution of one or more nucleotides, such as a single nucleotide polymorphism (SNP)) relative to a reference sequence (e.g., a wild type sequence). The term also encompasses the corresponding change in the complement of the nucleotide sequence, unless otherwise indicated. A nucleotide variation may be a somatic mutation or a germline polymorphism.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation. The terms “variation” and “mutation” may be used interchangeably.

The term “a nucleotide variation at a nucleotide position selected from Table 1” and grammatical variants thereof refer to a nucleotide variation in a LKB1 polynucleotide sequence at any of the nucleotide positions listed in Table 1, including but not limited to any of the nucleotide positions corresponding to the amino acid positions listed in column 1 of Table 1 and the specific nucleotide changes listed in column 2 of Table 1. For example and for purposes of illustration, with reference to column 1 in the third data row of Table 1, a nucleotide variation at any of the three nucleotide positions that correspond to amino acid 37 of a LKB1 polypeptide encompasses any change at one of those three nucleotide positions, including but not limited to the specific nucleotide change, i.e., the 109C>T substitution indicated in column 2. The term also encompasses the corresponding change in the complement of the nucleotide sequence, unless otherwise indicated.

The term “a nucleotide change selected from Table 1” and grammatical variants thereof refer to any of the specific nucleotide changes listed in column 2 of Table 1. For purposes of illustration, an example of a nucleotide change selected from Table 1 is the 109C>T substitution shown in the second column and third data row of Table 1.

The term “an amino acid variation at an amino acid position selected from Table 1” and grammatical variants thereof refer to an amino acid variation in a LKB1 amino acid sequence at any of the amino acid positions listed in column 1 of Table 1, including but not limited to the specific amino acid changes listed in column 3 of Table 1. For example and for purposes of illustration, with reference to column 1 in the fourth data row of Table 1, an amino acid variation at amino acid position 281 of LKB1 encompasses any change at that amino acid position, including but not limited to the specific amino acid change, i.e., the P281L substitution indicated in the fourth data row of column 3.

The term “an amino acid change selected from Table 1” and grammatical variants thereof refer to any of the specific amino acid changes listed in column 3 of Table 1. For purposes of illustration, an example of an amino acid change selected from Table 1 is the P281L substitution indicated in the fourth data row of column 3 or Table 1.

The term “LKB1 mutation,” as used herein, refers to one or more variations in the LKB1 gene that result in (a) significantly reduced or absent levels of LKB1 protein and/or (b) expression of a LKB1 protein with significantly reduced activity. The term “LKB1 mutation” includes but is not limited to nucleotide deletions that result in deletion of the entire LKB1 gene. A “mutated LKB1 gene” as used herein refers to a LKB1 gene comprising a LKB1 mutation. Levels of LKB1 protein are considered to be “significantly reduced or absent” when the levels of LKB1 in a cell, e.g., a tumor cell, are at 50% or lower than wild-type levels of the LKB1 protein in the same cell type, e.g., a normal cell corresponding to the same tissue type as the tumor cell. An LKB1 protein is considered to have “significantly reduced activity” when the kinase activity of the LKB1 protein is <50%, <40%, <30%, <20% or <10% of the kinase activity of wild-type LKB1 protein, as determined by a phosphorylation assay as described, e.g., in EP1633883 B1.

A cancer or tumor that “comprises a LKB1 mutation” refers to a cancer or tumor in which at least a portion of the cells of the cancer or tumor comprise a LKB1 mutation.

A “tumor sample” herein is a sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

A “fixed” tumor sample is one which has been histologically preserved using a fixative.

A “formalin-fixed” tumor sample is one which has been preserved using formaldehyde as the fixative.

An “embedded” tumor sample is one surrounded by a firm and generally hard medium such as paraffin, wax, celloidin, or a resin. Embedding makes possible the cutting of thin sections for microscopic examination or for generation of tissue microarrays (TMAs).

A “paraffin-embedded” tumor sample is one surrounded by a purified mixture of solid hydrocarbons derived from petroleum.

Herein, a “frozen” tumor sample refers to a tumor sample which is, or has been, frozen.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., PCR). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond may be used, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizing a nucleic acid polymerase to amplify or generate a specific piece of nucleic acid or to amplify or generate a specific piece of nucleic acid which is complementary to a particular nucleic acid.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “allele-specific oligonucleotide” refers to an oligonucleotide that hybridizes to a region of a target nucleic acid that comprises a nucleotide variation (generally a substitution). “Allele-specific hybridization” means that, when an allele-specific oligonucleotide is hybridized to its target nucleic acid, a nucleotide in the allele-specific oligonucleotide specifically base pairs with the nucleotide variation. An allele-specific oligonucleotide capable of allele-specific hybridization with respect to a particular nucleotide variation is said to be “specific for” that variation.

The term “allele-specific primer” refers to an allele-specific oligonucleotide that is a primer.

The term “primer extension assay” refers to an assay in which nucleotides are added to a nucleic acid, resulting in a longer nucleic acid, or “extension product,” that is detected directly or indirectly.

The term “allele-specific nucleotide incorporation assay” refers to a primer extension assay in which a primer is (a) hybridized to target nucleic acid at a region that is 3′ of a nucleotide variation and (b) extended by a polymerase, thereby incorporating into the extension product a nucleotide that is complementary to the nucleotide variation.

The term “allele-specific primer extension assay” refers to a primer extension assay in which an allele-specific primer is hybridized to a target nucleic acid and extended.

The term “allele-specific oligonucleotide hybridization assay” refers to an assay in which (a) an allele-specific oligonucleotide is hybridized to a target nucleic acid and (b) hybridization is detected directly or indirectly.

The term “5′ nuclease assay” refers to an assay in which hybridization of an allele-specific oligonucleotide to a target nucleic acid allows for nucleolytic cleavage of the hybridized probe, resulting in a detectable signal.

The term “assay employing molecular beacons” refers to an assay in which hybridization of an allele-specific oligonucleotide to a target nucleic acid results in a level of detectable signal that is higher than the level of detectable signal emitted by the free oligonucleotide.

The term “oligonucleotide ligation assay” refers to an assay in which an allele-specific oligonucleotide and a second oligonucleotide are hybridized adjacent to one another on a target nucleic acid and ligated together (either directly or indirectly through intervening nucleotides), and the ligation product is detected directly or indirectly.

The term “target sequence,” “target nucleic acid,” or “target nucleic acid sequence” refers generally to a polynucleotide sequence of interest in which a nucleotide variation is suspected or known to reside, including copies of such target nucleic acid generated by amplification.

The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., a lung cancer. “Diagnosis” may also refer to the classification of a particular type of cancer, e.g., by histology (e.g., a non small cell lung carcinoma), by molecular features (e.g., a lung cancer characterized by nucleotide and/or amino acid variation(s) in a particular gene or protein), or both.

The term “prediction” is used herein to refer to the likelihood that a patient will respond either favorably or unfavorably to a drug or set of drugs. In some embodiments, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer, colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

The terms “lung tumor” and “lung cancer” refer to any tumor of the lung, including but not limited to small-cell lung carcinoma and non-small cell lung carcinoma, the latter including but not limited to adenocarcinoma, squamous carcinoma, and large cell carcinoma.

The term “neoplasm” or “neoplastic cell” refers to an abnormal tissue or cell that proliferates more rapidly than corresponding normal tissues or cells and continues to grow after removal of the stimulus that initiated the growth.

A “lung tumor cell” or “lung cancer cell” refers to a cell from a lung tumor, either in vivo or in vitro, and encompasses cells derived from primary lung tumors or metastatic lung tumors, as well as cell lines derived from such cells.

As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “individual,” “subject” or “patient” is a vertebrate. In certain embodiments, the vertebrate is a mammal. Mammals include, but are not limited to, farm animals (such as cows), sport animals, pets (such as cats, dogs, and horses), primates (including human and non-human primates), and rodents (e.g., mice and rats). In certain embodiments, a mammal is a human.

An “effective amount” refers to an amount effective, at dosages and for periods of time necessary, to achieve the desired therapeutic or prophylactic result.

The term “therapeutically effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST), result in an objective response (including a partial response, PR, or complete respose, CR), improve survival (including overall survival and progression free survival) and/or improve one or more symptoms of cancer (e.g. as assessed by FOSI). Most preferably, the therapeutically effective amount of the drug is effective to improve progression free survival (PFS) and/or overall survival (OS).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks.

A “medicament” is an active drug to treat cancer, such as an EGFR inhibitor.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products.

The term “long-term” survival is used herein to refer to survival for at least 1 year, 5 years, 8 years, or 10 years following therapeutic treatment.

The term “increased resistance” to a particular therapeutic agent or treatment option, when used in accordance with the invention, means decreased response to a standard dose of the agent or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

The term “antagonist” is used in the broadest sense, and includes any molecule that partially or fully inhibits or neutralizes a biological activity of a polypeptide, such as EGFR, or that partially or fully inhibits the transcription or translation of a nucleic acid encoding the polypeptide. Exemplary antagonist molecules include, but are not limited to, antagonist antibodies, polypeptide fragments, oligopeptides, organic molecules (including small molecules), and anti-sense nucleic acids.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A “tumoricidal” agent causes destruction of tumor cells.

Herein, an “anti-tumor agent” refers to a drug used to treat cancer. Non-limiting examples of anti-tumor agents herein include chemotherapeutic agents, HER inhibitors, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

An “approved anti-tumor agent” is a drug used to treat cancer which has been accorded marketing approval by a regulatory authority such as the Food and Drug Administration (FDA) or foreign equivalent thereof.

A “HER inhibitor” is an agent which interferes with HER activation or function. Examples of HER inhibitors include HER antibodies (e.g. EGFR, HER2, HER3, or HER4 antibodies); EGFR-targeted drugs; small molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt. In some embodiments, the HER inhibitor is an antibody which binds to a HER receptor. In some embodiments, the HER inhibitor is a HER3 inhibitor. In some embodiments, the inhibitor is a multispecific HER inhibitor, e.g., a HER inhibitor, such as one which inhibits both HER3 and EGFR, HER3 and HER2, or HER3 and HER4. In such embodiments, the bispecific HER inhibitor is an antibody. In some embodiments, the HER inhibitor is a bispecific antibody that is specific for both HER3 and EGFR. Examples of such inhibitors are the multispecific antibodies described in US2010/0255010 (expressly incorporated herein by reference), including but not limited to the anti-EGFR/HER3 antibody “DL11f”.

As used herein, the term “EGFR inhibitor” refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or cetuximab; ERBITUX) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); mAb 806 or humanized mAb 806 (Johns et al., J. Biol. Chem. 279(29):30375-30384 (2004)); and humanized ICR62 antibodies including but not limited to GA201 (Gerdes et al. Clin. Cancer Res. 19(5):1126-1138 (2013) and antibodies described in U.S. Pat. No. 7,722,867 and WO2006/082515, expressly incorporated by reference herein. The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI-1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSAJ) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]6[5[[[2-methylsulfonyl)ethyl]amino]methyl]-2-furanyl]-4-quinazolinamine; Glaxo-SmithKline).

In some embodiments, “EGFR inhibitor” includes a multispecific HER inhibitor as described above that inhibits EGFR and one or more additional members of the HER family, e.g., HER2, HER3 or HER4. In such embodiments, an EGFR inhibitor is a bispecific HER inhibitor that binds EGFR and one other member of the HER family. In such embodiments, the bispecific HER inhibitor is an antibody. In such embodiments, the bispecific antibody is specific for both HER3 and EGFR. Examples of EGFR inhibitors include multispecific antibodies described in US2010/0255010 (expressly incorporated herein by reference), including but not limited to the anti-EGFR/HER3 antibody “DL11f”.

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVECJ, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVECJ); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca); and WO 1996/33980 (Zeneca).

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

A “small molecule” or “small organic molecule” is defined herein as an organic molecule having a molecular weight below about 500 Daltons.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At-211, Cu-67, Bi-212, and Pd-109.

An “isolated” biological molecule, such as a nucleic acid, polypeptide, or antibody, is one which has been identified and separated and/or recovered from at least one component of its natural environment.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (501 g/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength, and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

### II. Description of Certain Embodiments

Nucleotide and amino acid variations in LKB1 associated with tumor responsiveness to EGFR inhibitors are provided herein. These variations provide biomarkers for responsiveness to EGFR inhibitors.

A. Variations

Known variations in the LKB1 gene in primary tumors and cultured tumor cells are identified, for example, in the Catalogue of Somatic Mutations In Cancer (COSMIC, cancer.sanger.ac.uk/cancergenome/projects/cosmic/). Table 1 shows a list of LKB1 variations in the COSMIC database that have been identified in primary tumors and cultured tumor cells. The variations in Table 1 are listing in descending order of occurrences, and then by amino acid position along the LKB1 protein. The first column of the table lists the amino acid position of the variation in the LKB1 protein, the second column lists LKB1 coding sequence change resulting from the variation. The third column lists the LKB1 amino acid sequence change resulting from the variation. The fourth column lists the mutation ID, which is the identification number assigned to each variation. The fifth column lists the number of unique samples that have the variation. The last column lists the type of variation.

TABLE 1

| | | | Mutation | | |
|---|---|---|---|---|---|
| Amino acid | Coding sequence | Amino acid | ID | | |
| position | mutation | mutation | (COSM) | Count | Type |
| 354 | c.1062C>G | p.F354L | 21360 | 26 | substitution_missense |
| 1 | c.1_1302del1302 | p.0? | 27023 | 21 | deletion |
| 37 | c.109C>T | p.Q37* | 12925 | 13 | substitution_nonsense |
| 281 | c.842C>T | p.P281L | 21355 | 10 | substitution_missense |
| 170 | c.508C>T | p.Q170* | 20943 | 8 | substitution_nonsense |
| 194 | c.580G>T | p.D194Y | 20944 | 8 | substitution_missense |
| 281 | c.842delC | p.P281fs*6 | 12924 | 8 | deletion_frameshift |
| 220 | c.658C>T | p.Q220* | 13480 | 5 | substitution_nonsense |
| 57 | c.169delG | p.E57fs*7 | 21212 | 4 | deletion_frameshift |
| 156 | c.465_597del133 | p.Y156fs*87 | 27346 | 4 | deletion_frameshift |
| 264 | c.787_790delTTGT | p.F264fs*22 | 20857 | 4 | deletion_frameshift |
| 272 | c.816C>T | p.Y272Y | 29005 | 4 | substitution_synonymous |
| | c.734+1G>T | p.? | 51523 | 4 | unknown |
| 60 | c.180delC | p.Y60fs*1 | 27322 | 3 | deletion_frameshift |
| 70 | c.208G>T | p.E70* | 25846 | 3 | substitution_nonsense |
| 98 | c.291_464del174 | p.E98_G155del | 27344 | 3 | deletion_inframe |
| 120 | c.358G>T | p.E120* | 20875 | 3 | substitution_nonsense |
| 194 | c.580G>A | p.D194N | 25847 | 3 | substitution_missense |
| 199 | c.595G>T | p.E199* | 25229 | 3 | substitution_nonsense |
| 216 | c.647C>T | p.S216F | 25844 | 3 | substitution_missense |
| 282 | c.842_843insC | p.L282fs*3 | 25851 | 3 | insertion_frameshift |
| 332 | c.996G>A | p.W332* | 18652 | 3 | substitution_nonsense |
| | c.735−1G>T | p.? | 51522 | 3 | unknown |
| 32 | c.96C>G | p.T32T | 21378 | 2 | substitution_synonymous |
| 33 | c.97G>T | p.E33* | 95668 | 2 | substitution_nonsense |
| 53 | c.153delG | p.D53fs*11 | 27282 | 2 | deletion_frameshift |
| 56 | c.166G>T | p.G56W | 48784 | 2 | substitution_missense |
| 66 | c.196G>A | p.V66M | 21384 | 2 | substitution_missense |
| 159 | c.475C>T | p.Q159* | 27316 | 2 | substitution_nonsense |
| 165 | c.493G>T | p.E165* | 48902 | 2 | substitution_nonsense |
| 171 | c.511G>A | p.G171S | 21354 | 2 | substitution_missense |
| 217 | c.650delC | p.P217fs*70 | 20880 | 2 | deletion_frameshift |
| 223 | c.667G>T | p.E223* | 20870 | 2 | substitution_nonsense |
| 239 | c.717G>T | p.W239C | 333593 | 2 | substitution_missense |
| 281 | c.837delC | p.P281fs*6 | 20871 | 2 | deletion_frameshift |
| 304 | c.910C>G | p.R304G | 48789 | 2 | substitution_missense |
| 304 | c.910C>T | p.R304W | 29468 | 2 | substitution_missense |
| | c.291−2A>T | p.? | 49010 | 2 | unknown |
| | c.465_862del398 | p.? | 51203 | 2 | unknown |
| | c.465−1G>T | p.? | 21570 | 2 | unknown |
| 1 | c.2T>C | p.M1T | 20951 | 1 | substitution_missense |
| 1 | c.1_290del290 | p.? | 27015 | 1 | deletion_frameshift |
| 6 | c.17C>A | p.P6Q | 29463 | 1 | substitution_missense |
| 14 | c.40G>A | p.E14K | 21385 | 1 | substitution_missense |
| 16 | c.47_651del605 | p.E16fs*48 | 27325 | 1 | deletion_frameshift |
| 19 | c.56C>A | p.S19* | 29462 | 1 | substitution_nonsense |
| 26 | c.75_76CA>T | p.I26fs*25 | 27317 | 1 | complex |
| 36 | c.108C>A | p.Y36* | 20947 | 1 | substitution_nonsense |
| 37 | c.110A>T | p.Q37L | 48783 | 1 | substitution_missense |
| 41 | c.120_130del11 | p.K41fs*118 | 27318 | 1 | deletion_frameshift |
| 43 | c.126_149del24 | p.A43_L50del | 27284 | 1 | deletion_inframe |
| 43 | c.127_128insGG | p.A43fs*9 | 27319 | 1 | insertion_frameshift |
| 44 | c.130A>T | p.K44* | 20868 | 1 | substitution_nonsense |
| 44 | c.129delC | p.K44fs*7 | 27320 | 1 | deletion_frameshift |
| 48 | c.143delA | p.K48fs*3 | 27321 | 1 | deletion_frameshift |
| 48 | c.143_144>T | p.K48fs*3 | 26817 | 1 | complex |
| 49 | c.145T>G | p.Y49D | 20941 | 1 | substitution_missense |
| 50 | c.148_159del12 | p.L50_D53del | 51519 | 1 | deletion_inframe |
| 51 | c.152_153insCT | p.M51fs*14 | 22927 | 1 | insertion_frameshift |
| 52 | c.153_536del384 | p.G52_P179del | 27343 | 1 | deletion_inframe |
| 53 | c.152_153insG | p.D53fs*110 | 391251 | 1 | insertion_frameshift |
| 53 | c.157delG | p.D53fs*11 | 48969 | 1 | deletion_frameshift |
| 56 | c.166_178del13 | p.G56fs*4 | 48970 | 1 | deletion_frameshift |
| 56 | c.165_166insT | p.G56fs*107 | 25845 | 1 | insertion_frameshift |
| 56 | c.167G>T | p.G56V | 43187 | 1 | substitution_missense |
| 57 | c.167_168insTTCC | p.E57fs*107 | 166199 | 1 | insertion_frameshift |
| 57 | c.165delG | p.E57fs*7 | 392566 | 1 | deletion_frameshift |
| 57 | c.169G>T | p.E57* | 29464 | 1 | substitution_nonsense |
| 60 | c.180C>G | p.Y60* | 20874 | 1 | substitution_nonsense |
| 60 | c.? | p.Y60* | 133062 | 1 | substitution_nonsense |
| 60 | c.180C>A | p.Y60* | 48900 | 1 | substitution_nonsense |
| 61 | c.181delG | p.G61fs*3 | 405206 | 1 | deletion_frameshift |
| 62 | c.184A>T | p.K62* | 382838 | 1 | substitution_nonsense |
| 65 | c.193G>T | p.E65* | 20876 | 1 | substitution_nonsense |
| 69 | c.206C>A | p.S69* | 27315 | 1 | substitution_nonsense |
| 70 | c.209delA | p.E70fs*26 | 27323 | 1 | deletion_frameshift |
| 75 | c.224G>T | p.R75M | 327297 | 1 | substitution_missense |
| 77 | c.228delC | p.V77fs*19 | 27324 | 1 | deletion_frameshift |
| 78 | c.232A>G | p.K78E | 48785 | 1 | substitution_missense |
| 86 | c.256C>G | p.R86G | 29006 | 1 | substitution_missense |
| 87 | c.260G>A | p.R87K | 21075 | 1 | substitution_missense |
| 91 | c.271_272GG>TT | p.G91L | 48913 | 1 | substitution_missense |
| 98 | c.291_378del88 | p.? | 27016 | 1 | deletion_frameshift |
| 98 | c.291_597del307 | p.E98fs*87 | 27345 | 1 | deletion_frameshift |
| 100 | c.? | p.Q100E | 34162 | 1 | substitution_missense |
| 106 | c.318G>T | p.R106R | 21379 | 1 | substitution_synonymous |
| 106 | c.318G>A | p.R106R | 710012 | 1 | substitution_synonymous |
| 107 | c.320A>G | p.H107R | 29465 | 1 | substitution_missense |
| 108 | c.322A>T | p.K108* | 564718 | 1 | substitution_nonsense |
| 119 | c.357C>T | p.N119N | 20945 | 1 | substitution_synonymous |
| 123 | c.368A>G | p.Q123R | 25853 | 1 | substitution_missense |
| 123 | c.367C>T | p.Q123* | 380443 | 1 | substitution_nonsense |
| 135 | c.403G>C | p.G135R | 20942 | 1 | substitution_missense |
| 137 | c.411_412GG>TT | p.Q137_E138>H* | 1141538 | 1 | complex |
| 137 | c.409C>T | p.Q137* | 48901 | 1 | substitution_nonsense |
| 144 | c.432_433GG>TT | p.P144>? | 356375 | 1 | complex |
| 144 | c.431delC | p.P144fs*17 | 48971 | 1 | deletion_frameshift |
| 152 | c.454C>T | p.Q152* | 96526 | 1 | substitution_nonsense |
| 154 | c.462C>T | p.H154H | 327296 | 1 | substitution_synonymous |
| 155 | c.464_465GG>TTTGCT | p.G155fs*9 | 27350 | 1 | complex |
| 160 | c.479T>C | p.L160P | 21382 | 1 | substitution_missense |
| 163 | c.488G>A | p.G163D | 21352 | 1 | substitution_missense |
| 163 | c.487G>T | p.G163C | 25852 | 1 | substitution_missense |
| 165 | c.? | p.E165* | 210752 | 1 | substitution_nonsense |
| 168 | c.503A>G | p.H168R | 564715 | 1 | substitution_missense |
| 171 | c.513C>T | p.G171G | 327298 | 1 | substitution_synonymous |
| 174 | c.521A>G | p.H174R | 27283 | 1 | substitution_missense |
| 174 | c.522C>T | p.H174H | 474177 | 1 | substitution_synonymous |
| 175 | c.524A>T | p.K175M | 327299 | 1 | substitution_missense |
| 176 | c.527A>C | p.D176A | 564714 | 1 | substitution_missense |
| 176 | c.526G>T | p.D176Y | 27312 | 1 | substitution_missense |
| 176 | c.526G>C | p.D176H | 400197 | 1 | substitution_missense |
| 178 | c.532_536delAAGCC | p.K178fs*86 | 18562 | 1 | deletion_frameshift |
| 179 | c.536C>T | p.P179L | 51520 | 1 | substitution_missense |
| 179 | c.535C>T | p.P179S | 238600 | 1 | substitution_missense |
| 180 | c.539G>T | p.G180V | 96527 | 1 | substitution_missense |
| 181 | c.541A>T | p.N181Y | 564713 | 1 | substitution_missense |
| 181 | c.542A>T | p.N181I | 564712 | 1 | substitution_missense |
| 182 | c.544_546delCTG | p.L182del | 25843 | 1 | deletion_inframe |
| 188 | c.563delG | p.G188fs*99 | 27348 | 1 | deletion_frameshift |
| 191 | c.571A>T | p.K191* | 48903 | 1 | substitution_nonsense |
| 194 | c.579delC | p.D194fs*93 | 48972 | 1 | deletion_frameshift |
| 194 | c.581A>T | p.D194V | 20957 | 1 | substitution_missense |
| 194 | c.? | p.D194H | 133063 | 1 | substitution_missense |
| 194 | c.? | p.D194Y | 210753 | 1 | substitution_missense |
| 196 | c.587G>T | p.G196V | 48786 | 1 | substitution_missense |
| 197 | c.584_585insT | p.V197fs*69 | 25848 | 1 | insertion_frameshift |
| 199 | c.595G>C | p.E199Q | 27280 | 1 | substitution_missense |
| 199 | c.595G>A | p.E199K | 21359 | 1 | substitution_missense |
| 200 | c.598delG | p.A200fs*87 | 13481 | 1 | deletion_frameshift |
| 203 | c.607_610delCCGT | p.P203fs*83 | 22926 | 1 | deletion_frameshift |
| 204 | c.610_623del14 | p.F204fs*57 | 27326 | 1 | deletion_frameshift |
| 205 | c.613G>A | p.A205T | 20953 | 1 | substitution_missense |
| 208 | c.622G>A | p.D208N | 21356 | 1 | substitution_missense |
| 210 | c.630C>A | p.C210* | 20869 | 1 | substitution_nonsense |
| 212 | c.633delG | p.T212fs*75 | 26819 | 1 | deletion_frameshift |
| 214 | c.? | p.Q214* | 166351 | 1 | substitution_nonsense |
| 215 | c.644G>A | p.G215D | 21357 | 1 | substitution_missense |
| 216 | c.646T>C | p.S216P | 96336 | 1 | substitution_missense |
| 217 | c.649_650insG | p.P217fs*49 | 27281 | 1 | insertion_frameshift |
| 218 | c.650_651insC | p.A218fs*48 | 20858 | 1 | insertion_frameshift |
| 218 | c.654T>C | p.A218A | 377894 | 1 | substitution_synonymous |
| 219 | c.657C>T | p.F219F | 96221 | 1 | substitution_synonymous |
| 221 | c.662C>T | p.P221L | 377895 | 1 | substitution_missense |
| 223 | c.? | p.E223L | 133061 | 1 | substitution_missense |
| 231 | c.691T>C | p.F231L | 21383 | 1 | substitution_missense |
| 232 | c.? | p.S232fs*55 | 210754 | 1 | unknown |
| 235 | c.703A>T | p.K235* | 564711 | 1 | substitution_nonsense |
| 236 | c.704_705insA | p.V236fs*30 | 13581 | 1 | insertion_frameshift |
| 237 | c.709G>T | p.D237Y | 48787 | 1 | substitution_missense |
| 237 | c.709_709delG | p.D237fs*50 | 96530 | 1 | deletion_frameshift |
| 242 | c.725G>T | p.G242V | 48788 | 1 | substitution_missense |
| 242 | c.724G>T | p.G242W | 564710 | 1 | substitution_missense |
| 242 | c.724G>C | p.G242R | 25849 | 1 | substitution_missense |
| 246 | c.735_862del128 | p.Y246fs*3 | 27347 | 1 | deletion_frameshift |
| 251 | c.751G>C | p.G251R | 564708 | 1 | substitution_missense |
| 251 | c.752G>T | p.G251V | 564707 | 1 | substitution_missense |
| 255 | c.765_766CG>TT | p.F255>? | 374278 | 1 | complex |
| 265 | c.793G>T | p.E265* | 371077 | 1 | substitution_nonsense |
| 269 | c.802delG | p.K269fs*18 | 392578 | 1 | deletion_frameshift |
| 271 | c.810delG | p.S271fs*16 | 48973 | 1 | deletion_frameshift |
| 276 | c.827delG | p.G276fs*11 | 25850 | 1 | deletion_frameshift |
| 277 | c.829G>T | p.D277Y | 27313 | 1 | substitution_missense |
| 279 | c.835_836GG>TT | p.G279F | 85760 | 1 | substitution_missense |
| 281 | c.841_842>T | p.P281fs*6 | 28298 | 1 | complex |
| 285 | c.854T>A | p.L285Q | 25226 | 1 | substitution_missense |
| 287 | c.859A>T | p.K287* | 332311 | 1 | substitution_nonsense |
| 290 | c.870T>A | p.L290L | 20952 | 1 | substitution_synonymous |
| 294 | c.882G>A | p.P294P | 327300 | 1 | substitution_synonymous |
| 294 | c.879_880insA | p.P294fs*24 | 29466 | 1 | insertion_frameshift |
| 297 | c.891G>C | p.R297S | 96528 | 1 | substitution_missense |
| 298 | c.894C>A | p.F298L | 29467 | 1 | substitution_missense |
| 308 | c.923G>T | p.W308L | 26041 | 1 | substitution_missense |
| 308 | c.? | p.W308L | 87888 | 1 | substitution_missense |
| 312 | c.936delA | p.K312fs*24 | 20948 | 1 | deletion_frameshift |
| 314 | c.941C>A | p.P314H | 21353 | 1 | substitution_missense |
| 317 | c.949G>T | p.E317* | 28292 | 1 | substitution_nonsense |
| 320 | c.957_958AG>T | p.V320fs*16 | 20958 | 1 | complex |
| 324 | c.971C>T | p.P324L | 21380 | 1 | substitution_missense |
| 327 | c.979_980insAG | p.D327fs*10 | 48942 | 1 | insertion_frameshift |
| 328 | c.984C>T | p.T328T | 20946 | 1 | substitution_synonymous |
| 347 | c.1039_1040insG | p.A347fs*13 | 27349 | 1 | insertion_frameshift |
| 350 | c.1050C>T | p.D350D | 21381 | 1 | substitution_synonymous |
| 367 | c.1100C>T | p.T367M | 21358 | 1 | substitution_missense |
| 389 | c.1165G>A | p.A389T | 48790 | 1 | substitution_missense |
| 403 | c.1208A>T | p.K403I | 327302 | 1 | substitution_missense |
| 409 | c.1225C>T | p.R409W | 25854 | 1 | substitution_missense |
| 419 | c.1257C>T | p.S419S | 20956 | 1 | substitution_synonymous |
| 425 | c.1274G>A | p.R425H | 327301 | 1 | substitution_missense |
| 426 | c.1276C>T | p.R426W | 27314 | 1 | substitution_missense |
| | c.921−10G>A | p.? | 21386 | 1 | unknown |
| | c.598−2A>T | p.? | 25858 | 1 | unknown |
| | c.597+1G>T | p.? | 49004 | 1 | unknown |
| | c.863−2A>T | p.? | 401786 | 1 | unknown |
| | c.598−13del22 | p.? | 49012 | 1 | unknown |
| | c.?_?insG | p.?fs | 20877 | 1 | insertion_frameshift |
| | c.921−1G>A | p.? | 49008 | 1 | unknown |
| | c.465−1G>A | p.? | 25855 | 1 | unknown |
| | c.?_?del? | p.? | 20950 | 1 | unknown |
| | c.735−2A>T | p.? | 25856 | 1 | unknown |
| | c.598−1G>T | p.? | 51521 | 1 | unknown |
| | c.1_378del378 | p.? | 51202 | 1 | unknown |
| | c.379_433del55 | p.? | 25859 | 1 | unknown |
| | c.? | p.D53fs*11 | 133064 | 1 | unknown |
| | c.1109_1302del194 | p.? | 51227 | 1 | unknown |
| | c.?_?del? | p.? | 20955 | 1 | unknown |
| | c.291−1G>T | p.? | 564719 | 1 | unknown |
| | c.?_?del? | p.? | 20954 | 1 | unknown |
| | c.1_597del597 | p.? | 51201 | 1 | unknown |
| | c.?_?del? | p.? | 20879 | 1 | unknown |
| | c.734+5G>T | p.? | 564709 | 1 | unknown |
| | c.465−2A>G | p.? | 21387 | 1 | unknown |
| | c.290+2T>G | p.? | 25857 | 1 | unknown |
| | c.291−11_305del26 | p.? | 29469 | 1 | unknown |

LKB1 amino acids are numbered according to their positions in the translated cDNA sequence. Where a nucleotide substitution resulted in a stop codon (i.e., a nonsense mutation), the corresponding amino acid change is indicated by a “*” (see, e.g., Position 37 variation at third data row, indicating an amino acid change of “Q37*”). Where a nucleotide insertion or deletion results in a frame shift, the corresponding amino acid change is indicated by “fs*” and then a number, which indicates the number of amino acids after the frameshift before a stop codon (see, e.g., “p.P281fs*6” at position 281.). Deletions of portions of the coding sequence are indicated by the first nucleotide of the deletion, underscore (_), then the last nucleotide of the deletion, and the number of deleted nucleotides follows the word “del” (see, e.g., “c.465_{—}597de1133” in data row 10). Mutations in splice junctions are indicated by “+” (splice donor) and “−” (splice acceptor), where the number following the + or − indicates the position relative to the GT (donor site, where G is +1 and T is +2, etc.) or to the AG (acceptor site, where G is −1, A is −2, etc.). In some instances, LKB1 genes comprising splice site mutations may not express LKB1 protein (indicated by “p.?”).

Table 2 shows a nonlimiting exemplary list of tumors in which LKB1 mutations have been identified in the COSMIC database. The first column shows the primary tissue from which the tumor originated. The second column indicates the number of unique samples of that type having LKB1 variations. The third column indicates the total number of unique samples of that type. The fourth column indicates the percentage of tumors of that type that have been identified as having LKB1 variations.

TABLE 2

| | Unique | Total | |
|---|---|---|---|
| | mutated | unique | % |
| Primary tissue | samples | samples | Mutated |
| gastrointestinal_tract_(site_indeterminate) | 5 | 21 | 23.81 |
| cervix | 29 | 214 | 13.55 |
| small_intestine | 1 | 10 | 10 |
| lung | 236 | 2809 | 8.4 |
| skin | 15 | 306 | 4.9 |
| biliary_tract | 1 | 39 | 2.56 |
| testis | 1 | 45 | 2.22 |
| stomach | 9 | 475 | 1.89 |
| large_intestine | 14 | 1095 | 1.28 |
| liver | 1 | 81 | 1.23 |
| pancreas | 6 | 557 | 1.08 |
| oesophagus | 1 | 111 | 0.9 |
| prostate | 2 | 336 | 0.6 |
| upper_aerodigestive_tract | 1 | 176 | 0.57 |
| urinary_tract | 1 | 176 | 0.57 |
| haematopoietic_and_lymphoid_tissue | 4 | 803 | 0.5 |
| breast | 3 | 615 | 0.49 |
| kidney | 2 | 487 | 0.41 |
| ovary | 3 | 827 | 0.36 |

Although the variations described herein were identified in the tumor types indicated in Table 2, other types of cancers may be routinely screened to determine whether any of these variations occur in those cancers. The methods of the present invention are applicable to any cancer comprising a variation in LKB1, whether or not the variation is one that is listed in Table 1.

A nucleotide variation, according to any of the above methods, may be a somatic mutation or a germline polymorphism.

B. Compositions

In some embodiments, an allele-specific oligonucleotide is provided that hybridizes to a region of a LKB1 polynucleotide comprising a nucleotide variation (e.g., a substitution). In some embodiments, the nucleotide variation is at a nucleotide position selected from Table 1. In some embodiments, the nucleotide variation is a nucleotide change selected from Table 1. The allele-specific oligonucleotide, when hybridized to the region of the LKB1 polynucleotide, comprises a nucleotide that base pairs with the nucleotide variation. In some embodiments, the complement of an allele-specific oligonucleotide is provided. In some embodiments, a microarray comprises one or more allele-specific oligonucleotides and/or their complements. In some embodiments, an allele-specific oligonucleotide or its complement is an allele-specific primer.

An allele-specific oligonucleotide can be used in conjunction with a control oligonucleotide that is identical to the allele-specific oligonucleotide, except that the nucleotide that specifically base pairs with the nucleotide variation is replaced with a nucleotide that specifically base pairs with the corresponding nucleotide present in the wild type LKB1 polynucleotide. Such oligonucleotides may be used in competitive binding assays under hybridization conditions that allow the oligonucleotides to distinguish between a LKB1 polynucleotide comprising a nucleotide variation and a LKB1 polynucleotide comprising the corresponding wild type nucleotide. Using routine methods based on, e.g., the length and base composition of the oligonucleotides, one skilled in the art can arrive at suitable hybridization conditions under which (a) an allele-specific oligonucleotide will preferentially bind to a LKB1 polynucleotide comprising a nucleotide variation relative to a wild type LKB1 polynucleotide, and (b) the control oligonucleotide will preferentially bind to a wild type LKB1 polynucleotide relative to a LKB1 polynucleotide comprising a nucleotide variation. Exemplary conditions include conditions of high stringency, e.g., hybridization conditions of 5× standard saline phosphate EDTA (SSPE) and 0.5% NaDodSO₄ (SDS) at 55° C., followed by washing with 2×SSPE and 0.1% SDS at 55° C. or room temperature.

In some embodiments, an isolated polynucleotide provided herein is detectably labeled, e.g., with a radioisotope, a fluorescent agent, or a chromogenic agent. In another embodiment, an isolated polynucleotide is a primer. In another embodiment, an isolated polynucleotide is an oligonucleotide, e.g., an allele-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Oligonucleotides as provided herein are useful, e.g., as hybridization probes for the detection of nucleotide variations.

In another aspect, a binding agent is provided that preferentially binds to a LKB1 comprising an amino acid variation, relative to a wild-type LKB1. In some embodiments, the amino acid variation is at an amino acid position selected from FIG. 2. In some embodiments, the amino acid variation is an amino acid change selected from FIG. 2. In some embodiments, the binding agent is an antibody.

In some embodiments, diagnostic kits are provided. In some embodiments, a kit comprises any of the foregoing polynucleotides. In some embodiments, a kit further comprises an enzyme. In some embodiments, the enzyme is at least one enzyme selected from a nuclease, a ligase, and a polymerase. In some embodiments, a kit comprises any of the foregoing binding agents.

C. Therapeutic and Diagnostic Methods

Somatic mutations in LKB1 have been found in many sporadic cancers, including, for example, non-small cell lung cancer and the cancers shown in Table 2. The present inventors have found that lung and pancreatic tissue comprising an inactivated LKB1 gene shows substantially more growth in the presence of EGF than wild-type tissue. Further, the EGF-induced growth in the tissue comprising an inactivated LKB1 gene was effectively inhibited by an EGFR inhibitor.

Accordingly, in some embodiments, a method for predicting the responsiveness of a cancer to an EGFR inhibitor is provided, the method comprising determining the presence of a LKB1 mutation in the cancer, wherein the presence of the LKB1 mutation in the cancer indicates the cancer will respond to treatment with the EGFR inhibitor. In some embodiments, a method of identifying a cancer patient who is likely to benefit from an EGFR inhibitor is provided, comprising determining whether the patient's cancer comprises a LKB1 mutation, wherein the presence of the LKB1 mutation indicates that the cancer patient will likely benefit from the EGFR inhibitor. In some embodiments, a method of selecting a therapy for a cancer patient is provided, comprising (a) determining whether the patient's cancer comprises a LKB1 mutation; and (b) if the patient's cancer comprises a LKB1 mutation, selecting an EGFR inhibitor for the therapy.

In some embodiments, the LKB1 mutation comprises a variation in a LKB1 polynucleotide. Exemplary variations include, but are not limited to, insertions, deletions, inversions, and substitutions, and may occur in the LKB1 coding sequence or in the noncoding regions of the gene. In some embodiments, the variation in the LKB1 polynucleotide is a nucleotide variation at a nucleotide position selected from Table 1. In some embodiments, the variation in the LKB1 polynucleotide is a nucleotide change selected from Table 1. In some embodiments, the LKB1 mutation is a deletion of the LKB1 gene.

Variations in the LKB1 polynucleotide may result in variations in the LKB1 polypeptide. Such variations include, but are not limited to, insertions, substitutions, deletions, and truncations. In some embodiments, the variation in the LKB1 polypeptide is a variation at an amino acid position selected from Table 1. In some embodiments, the variation in the LKB1 polypeptide is an amino acid change selected from Table 1.

In some embodiments, the cancer is selected from a large cell carcinoma, carcinoid tumor, tumor of neuroendrocrine origin, head and neck squamous cell carcinoma (HNSCC), colorectal cancer, cervical cancer, melanoma, skin cancer, leiomyoma, gastric cancer, glioblastoma, ovarian cancer, small cell lung cancer (SCLC), non-small cell lung cancer (NSCLC), pancreatic cancer, esophageal cancer, gastric cancer and thyroid cancer. In some embodiments, the cancer is selected from the cancers listed in Table 2. In some embodiments, the cancer is selected from lung cancer, pancreatic cancer, colorectal cancer, and head and neck cancer.

Methods of determining presence of LKB1 mutations in a cancer (i.e. from a sample taken from cancer, or from a cell isolated from the cancer by any means, such as by a biopsy or as a circulating cancer cell) are known in the art. For example, assays for detection of specific mutations in the LKB1 gene, using real-time PCR are known (available from Qiagen, Valencia, Calif.).

A nucleic acid, may be e.g., genomic DNA, RNA transcribed from genomic DNA, or cDNA generated from RNA. A nucleic acid may be derived from a vertebrate, e.g., a mammal. A nucleic acid is said to be “derived from” a particular source if it is obtained directly from that source or if it is a copy of a nucleic acid found in that source.

Variations in nucleic acids and amino acid sequences may be detected by certain methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. see, e.g., Wu et al., Genomics 4:560-569 (1989). In addition, a technique known as allele-specific PCR can also be used to detect variations (e.g., substitutions). see, e.g., Ruano and Kidd (1989) Nucleic Acids Research 17:8392; McClay et al. (2002) Analytical Biochem. 301:200-206. In certain embodiments of this technique, an allele-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) allele-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) allele-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′ nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl. Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can be a portion of the target nucleic acid, provided it encompasses the position suspected of having a variation.

In a similar manner, DNA probes can be used to detect mismatches, for example through enzymatic or chemical cleavage. see, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. see, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. see, e.g., Orita et al., Proc. Natl. Acad. Sci. USA 86:2766-2770, 1989, and Genomics, 5:874-879, 1989.

The invention also provides a variety of compositions suitable for use in performing methods of the invention. For example, the invention provides arrays that can be used in such methods. In some embodiments, an array of the invention comprises individual or collections of nucleic acid molecules useful for detecting variations. For instance, an array of the invention may comprise a series of discretely placed individual allele-specific oligonucleotides or sets of allele-specific oligonucleotides. Several techniques are well-known in the art for attaching nucleic acids to a solid substrate such as a glass slide. One method is to incorporate modified bases or analogs that contain a reactive moiety that is capable of attachment to a solid substrate, such as an amine group, a derivative of an amine group, or another group with a positive charge, into nucleic acid molecules that are synthesized. The synthesized product is then contacted with a solid substrate, such as a glass slide coated with an aldehyde or other reactive group. The aldehyde or other reactive group will form a covalent link with the reactive moiety on the amplified product, which will become covalently attached to the glass slide. Other methods, such as those using amino propryl silican surface chemistry are also known in the art.

The presence of a LKB1 mutation in a cancer, according to any of the above methods, can be determined using any suitable biological sample obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Variations in target nucleic acids (or encoded polypeptides) may be detected from a tumor sample or from other body samples such as urine, sputum or serum. Cancer cells are sloughed off from tumors and appear in such body samples. By screening such body samples, a simple early diagnosis can be achieved for diseases such as cancer. In addition, the progress of therapy can be monitored more easily by testing such body samples for variations in target nucleic acids (or encoded polypeptides). Additionally, methods for enriching a tissue preparation for tumor cells are known in the art. For example, the tissue may be isolated from paraffin or cryostat sections. Cancer cells may also be separated from normal cells by flow cytometry or laser capture microdissection.

In some embodiments, methods of treating a mammal with a cancer comprising a LKB1 mutation are provided, comprising administering to the mammal a therapeutically effective amount of an EGFR inhibitor.

In some embodiments, methods of treating a cancer in a mammal are provided, comprising (a) determining whether the cancer comprises a LKB1 mutation; and (b) if the cancer comprises a LKB1 mutation, administering to a mammal a therapeutically effective amount of an EGFR inhibitor.

In some embodiments, methods of treating a cancer comprising a LKB1 mutation are provided, wherein the method comprises administering a therapeutically effective amount of an EGFR inhibitor to a mammal with the cancer, wherein prior to administration of the EGFR inhibitor, the cancer was determined to have a LKB1 mutation.

Another aspect of the invention herein provides a method for treating a mammal with a type of cancer that exhibits a mutation in the LKB1 gene, comprising administering to the mammal a therapeutically effective amount of an EGFR inhibitor.

In any of the foregoing embodiments, the mammal may be a human.

In some embodiments, the EGFR inhibitor is an antibody that binds EGFR. In some embodiments, the EGFR inhibitor is a small molecule that binds EGFR. In some embodiments, the EGFR inhibitor is selected from erlotinib, cetuximab, panitumumab, lapatinib, DL11f, and GA201.

The EGFR inhibitor is administered to a mammal (such as a human patient) in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

For the prevention or treatment of cancer, the dose of the EGFR inhibitor, will depend on the type of cancer to be treated, as defined above, the severity and course of the cancer, whether the antibody is administered for preventive or therapeutic purposes, previous therapy, the patient's clinical history and response to the drug, and the discretion of the attending physician.

In some embodiments, a fixed dose of inhibitor is administered. The fixed dose may suitably be administered to the patient at one time or over a series of treatments. Where a fixed dose is administered, preferably it is in the range from about 20 mg to about 2000 mg of the inhibitor. For example, the fixed dose may be approximately 420 mg, approximately 525 mg, approximately 840 mg, or approximately 1050 mg of the inhibitor.

Where a series of doses are administered, these may, for example, be administered approximately every week, approximately every 2 weeks, approximately every 3 weeks, or approximately every 4 weeks, but preferably approximately every 3 weeks. The fixed doses may, for example, continue to be administered until disease progression, adverse event, or other time as determined by the physician. For example, from about two, three, or four, up to about 17 or more fixed doses may be administered.

In some embodiments, one or more loading dose(s) of the antibody are administered, followed by one or more maintenance dose(s) of the antibody. In another embodiment, a plurality of the same dose are administered to the patient.

While the EGFR inhibitor may be administered as a single anti-tumor agent, the patient is optionally treated with a combination of the inhibitor, and one or more chemotherapeutic agent(s).

Other therapeutic agents that may be combined with the inhibitor and/or chemotherapeutic agent include any one or more of: a second, different HER inhibitor, HER dimerization inhibitor (for example, a growth inhibitory HER2 antibody such as trastuzumab, or a HER2 antibody which induces apoptosis of a HER2-overexpressing cell, such as 7C2, 7F3 or humanized variants thereof); an antibody directed against a different tumor associated antigen, such as EGFR, HER3, HER4; anti-hormonal compound, e.g., an anti-estrogen compound such as tamoxifen, or an aromatase inhibitor; a cardioprotectant (to prevent or reduce any myocardial dysfunction associated with the therapy); a cytokine; an EGFR-targeted drug (such as TARCEVA®, IRESSA®, VECTIBIX®, or ERBITUX®); an anti-angiogenic agent (especially bevacizumab sold by Genentech under the trademark AVASTIN™); a tyrosine kinase inhibitor; a COX inhibitor (for instance a COX-1 or COX-2 inhibitor); non-steroidal anti-inflammatory drug, celecoxib (CELEBREX®); farnesyl transferase inhibitor (for example, Tipifarnib/ZARNESTRA® R115777 available from Johnson and Johnson or Lonafarnib SCH66336 available from Schering-Plough); antibody that binds oncofetal protein CA 125 such as Oregovomab (MoAb B43.13); HER2 vaccine (such as HER2AutoVac vaccine from Pharmexia, or APC8024 protein vaccine from Dendreon, or HER2 peptide vaccine from GSK/Corixa); another HER targeting therapy (e.g. trastuzumab, cetuximab, ABX-EGF, EMD7200, gefitinib, erlotinib, CP724714, CI1033, GW572016, IMC-11F8, TAK165, etc); Raf and/or ras inhibitor (see, for example, WO 2003/86467); doxorubicin HCl liposome injection (DOXIL®); topoisomerase I inhibitor such as topotecan; taxane; HER2 and EGFR dual tyrosine kinase inhibitor such as lapatinib/GW572016; TLK286 (TELCYTA®); EMD-7200; a medicament that treats nausea such as a serotonin antagonist, steroid, or benzodiazepine; a medicament that prevents or treats skin rash or standard acne therapies, including topical or oral antibiotic; a medicament that treats or prevents diarrhea; a body temperature-reducing medicament such as acetaminophen, diphenhydramine, or meperidine; hematopoietic growth factor, etc.

Suitable dosages for any of the above coadministered agents are those presently used and may be lowered due to the combined action (synergy) of the agent and inhibitor.

In addition to the above therapeutic regimes, the patient may be subjected to surgical removal of cancer cells and/or radiation therapy.

Where the inhibitor is an antibody, preferably the administered antibody is a naked antibody. However, the inhibitor administered may be conjugated with a cytotoxic agent. Preferably, the conjugated inhibitor and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases and DNA endonucleases.

The present application contemplates administration of the inhibitor by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

D. Articles of Manufacture

In some embodiments, an article of manufacture containing materials useful for the treatment of the diseases or conditions described above is provided. The article of manufacture comprises a container and a label or package insert on or associated with the container. Suitable containers include, for example, bottles, vials, syringes, etc. The containers may be formed from a variety of materials such as glass or plastic. The container holds or contains a composition which is effective for treating the disease or condition of choice and may have a sterile access port (for example the container may be an intravenous solution bag or a vial having a stopper pierceable by a hypodermic injection needle). At least one active agent in the composition is an EGFR inhibitor.

The article of manufacture may further comprise a second container comprising a pharmaceutically-acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

The kits and articles of manufacture of the present invention also include information, for example in the form of a package insert or label, indicating that the composition is used for treating cancer where the patient's cancer comprises an LKB1 mutation. The insert or label may take any form, such as paper or on electronic media such as a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the EGFR inhibitor may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

In a specific embodiment of the invention, an article of manufacture is provided comprising, packaged together, a pharmaceutical composition comprising a the EGFR inhibitor, in a pharmaceutically acceptable carrier and a label stating that the inhibitor or pharmaceutical composition is indicated for treating a patient with a type of cancer which is able to respond to a the EGFR inhibitor, wherein the patient's cancer comprises a mutation in the LKB1 gene as described herein.

In an optional embodiment of this aspect, the article of manufacture herein may further comprise a container comprising a second medicament, wherein the EGFR inhibitor is a first medicament, and which article further comprises instructions on the package insert for treating the patient with the second medicament, in an effective amount. The second medicament may be any of those set forth above.

The package insert is on or associated with the container. Suitable containers include, for example, bottles, vials, syringes, etc. The containers may be formed from a variety of materials such as glass or plastic. The container holds or contains a composition that is effective for treating cancer type may have a sterile access port (for example the container may be an intravenous solution bag or a vial having a stopper pierceable by a hypodermic injection needle). At least one active agent in the composition is the EGFR inhibitor. The label or package insert indicates that the composition is used for treating cancer in a subject eligible for treatment with specific guidance regarding dosing amounts and intervals of inhibitor and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

### III. Examples

A. Materials and Methods

### Lkb1 Vector Construction

The construct for targeting the C57BL/6 Stk11 (LKB1) locus was made using recombineering (Warming et al., 2005, Nucl. Acids Res. 33: e36). After retrieving a genomic fragment containing Lkb1 from a C57BL/6 BAC (RP23 library) into pBlight-TK, the sequence GTG-ATG-GAG-TAC-TGC-GTA within exon3 was replaced with GTT-GGG-GAA-TAT-TGC-GTA to change Met129 to a Glycine and replace a ScaI with an SspI site. A neomycin cassette flanked by loxp was then introduced into intron2. The final vector was confirmed by DNA sequencing and linearized. C57BL/6 C2 embryonic stem cells were targeted with standard methods and positive clones were transfected with Cre to remove the neomycin cassette. The modified embryonic stem cells were then injected into blastocysts and germline transmission was obtained by crossing the chimeras with C57BL6 females.

### Explant Cultures

Pancreas and individual lobes of the lungs were dissected from embryos harvested from timed pregnancy setups. Embryonic explants were cultured on 12 mm Transwell® with 0.4 μm polyester membrane insert (Corning, Tewsbury, Mass.) at the air-liquid interface in DME media with 10% fetal calf serum, glutamine and penicillin-streptomycin. For longer-term cultures (>3 days), media was changed daily.

For mesenchyme-free cultures, lungs were dissected and incubated in Collagenase/Dispase (1 mg/ml for each) for 10-15 minutes at room temperature. Epithelial tissue was removed by mechanical dissection, transferred to the Transwell® plates, and covered with 5-10 μl of 1:1 Matrigel:DME. After Matrigel was solidified, 400 μl media (DME/10% FCS/glutamine/pen-strep+200 ng/ml FGF7 (Life Technologies, Carlsbad, Calif.), FGF1 (Life Technologies), or EGF (Life Technologies)) was added to top and bottom wells. Erlotinib (Tarceva®, 1 μM hydrochloride salt; OSI Pharmaceuticals) or DMSO was added as indicated.

### Whole Mount Immunofluorescence

Embryonic explants were fixed in 4% paraformaldehyde, permeabilized in PBS with 2% BSA, 0.1% saponin or 0.1% Triton-X100. After washing, tissues were stained with anti-E-cadherin (Santa Cruz). Explants were mounted and imaged with a Leica SP5 laser confocal microscope. Images shown are representative of multiple independent experiments (n≧5) with littermate explants that were cultured, stained and imaged under substantially the same conditions and settings.

### Western Blot

Tissues were lysed using PhosphoSafe™ Extraction Reagent (EMD Millipore) and sonication. Protein concentrations of tissue and cell lysates were determined by BCA assay (Pierce). Samples in LDS Sample Buffer+β-mercaptoethanol were heated to 95° C. for 5 minutes before separation by electrophoresis using NuPAGE® 4-12% Bis Tris gels (Invitrogen). Proteins were transferred to nitrocellulose membranes overnight, membranes were blocked in Tris-buffered saline (TBS) buffer with 0.1% Tween and 5% Blotto before incubating overnight at 4° C. with primary antibodies in TBS buffer with 0.1% Tween and 2% BSA. Primary antibodies (EGF Receptor (D38B1) XP® Rabbit mAb, Phospho-EGF Receptor (Tyr845) (D63B4) Rabbit mAb, Phospho-EGF Receptor (Tyr1173) (53A5) Rabbit mAb, Cyclin D1 (92G2) Rabbit mAb, all from Cell Signaling Technology; and anti-actin antibody) were detected with HRP-conjugated secondary antibodies and ECL reagents (Pierce). Experiments shown are representative of multiple independent experiments (n≧3).

B. Results

Homozygous germline mutations in LKB1 in mice are early embryonic lethal. Therefore, a chemical genetic approach was used to design an allele that can be specifically inhibited by the addition of a cell permeable, non-hydrolyzable ATP analogue (NMPP1) (Bishop et al., 2000, Nature, 407: 393-401; Bishop at al., 2000, Ann. Rev. Biophys. Biomol. Struct. 29: 557-606). This allele (Lkb1^{MG}) was “knocked-in” to the endogenous LKB1 locus to generate a genetically engineered mouse line that has been described in detail elsewhere (Lo et al., 2012, J. Cell. Biol., 199: 1117-1130). Lung and pancreas tissues from Lkb1^{MG/MG} embryos and wild-type littermates were dissected and grown in vitro for several days. Lung cultures were grown both as whole mount explants, and “mesenchyme-free” where most of the mesenchyme and other tissue types were stripped from the lung epithelium and the epithelium was cultured in Matrigel (Lu et al., 2005, J. Biol. Chem., 280: 4834-4841). During this time, the response of these tissues to growth factors was assessed.

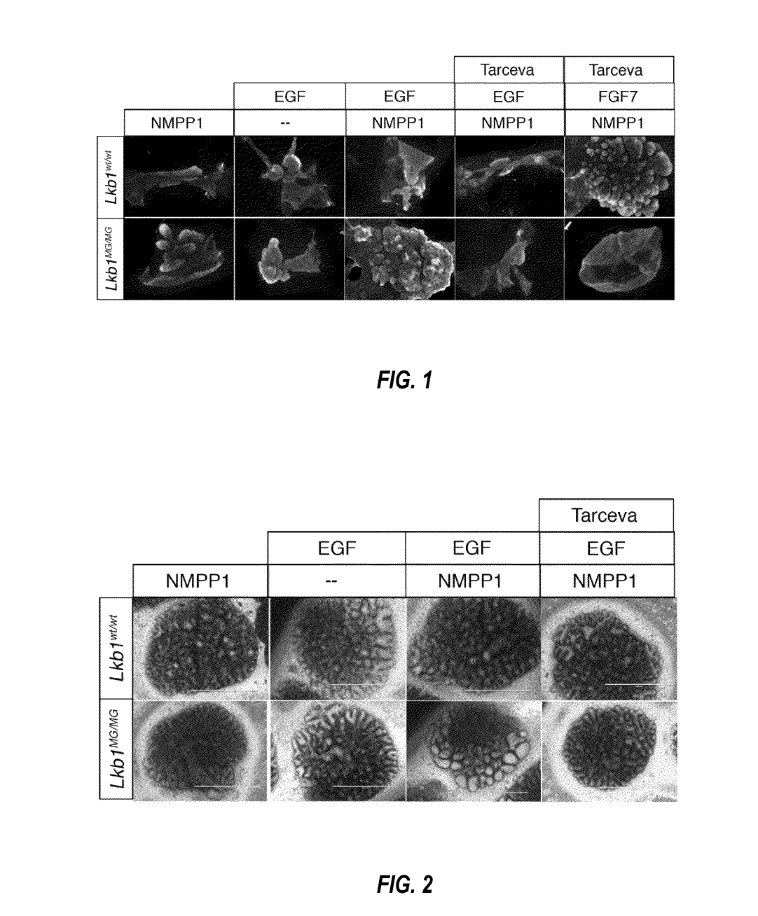

Addition of EGF (100 ng/ml) in the presence of NMPP1 inhibitor to mesenchyme free explants derived from Lkb1^{wt/wt} resulted in a modest increase in growth compared to no growth factor. In contrast, addition of EGF to Lkb1^{MG/MG} in the presence of NMPP1 resulted in substantial growth (FIG. 1), indicating that inhibition of LKB1 in mesenchyme free explants resulted in increased responsiveness to EGF. Similar results were seen in whole mount cultures (FIG. 2). In this case, addition of EGF resulted in substantial enlargement of the terminal buds of the cultured Lkb1^{MG/MG} lungs and only a modest increase in size in the LKB1^{wt/wt} lungs. In both the whole mount and mesenchyme free cultures, the EGF-induced growth was inhibited by addition of erlotinib (Tarceva®; 1 μM). In contrast, addition of fibroblast growth factor (FGF)-1 or FGF-7 resulted in robust growth of mesenchyme free lung cultures derived from both LKB1^{wt/wt} and LKB1^{MG/MG} in the presence and absence of NMPP1, although the epithelial tissue structures displayed less branching when LKB1 was inhibited.

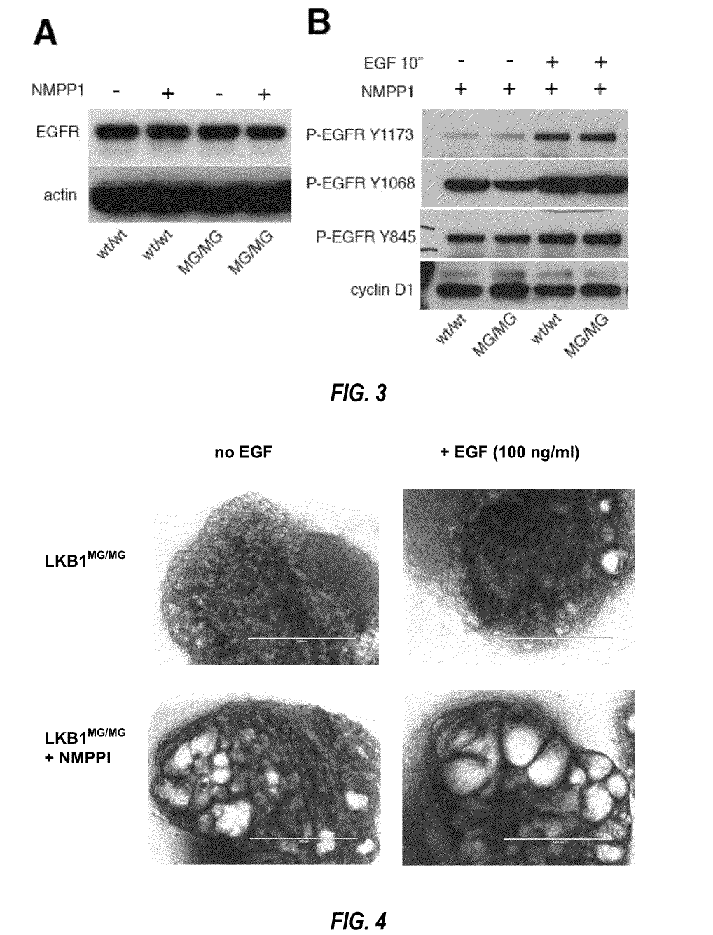

To assess whether this change in responsiveness was due to differential levels of EGFR expression, EGFR protein levels were measured by Western blotting. EGFR levels were found to be similar in both LKB1^{wt/wt} and LKB1^{MG/MG} lungs in the presence or absence of NMPP1. Furthermore, phosphorylation of EGFR at sites Y1173, Y1068, and Y845, which occurs within 10 minutes of addition of EGF, appeared unchanged (FIG. 3). Thus, the responsiveness of the cultures to EGF was not a result of increased EGFR expression.

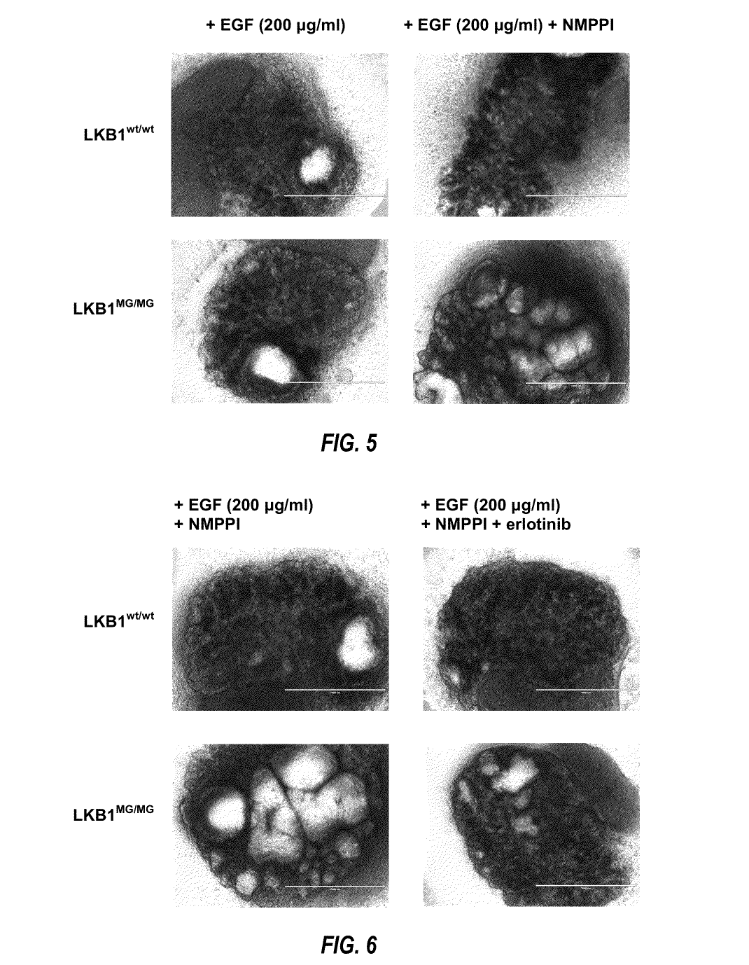

A similar role for LKB1 in modulating EGF signaling was identified in pancreas. Previously, we have shown that inhibition of Lkb1 kinase activity in pancreatic explants harvested from late stage embryos dramatically promotes cyst formation (Lo et al., 2012, J. Cell. Biol., 199: 1117-1130). In this experimental system, the addition of EGF (100 ng/ml) accelerates the development of the cystic phenotype induced by Lkb1 inhibition (FIG. 4). However, the cysts that develop in the presence and absence of EGF are morphologically indistinguishable, and in the absence of Lkb1 inhibition, EGF does not by itself promote cysts (FIG. 5). In addition, pancreatic cyst formation appears to require EGFR signaling since erlotinib (Tarceva®; 1 μM) inhibits the development of the cystic phenotype (FIG. 6).

We have shown that LKB1 activity alters the responsiveness of lung and pancreatic epithelial cells to EGF. Since EGF signaling is known to be a critical pathway in some cancers, LKB1 mutation status should be informative in assessing the vulnerability or resistance of this pathway to certain targeted therapies, and in particular, EGFR-targeted therapies. As shown by the above experiments, LKB1 mutations in cancer would be predicted to increase the responsiveness of the cancer to EGF, and therefore such cancer would be predicted to respond well to an EGFR inhibitor, such as those described herein.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

### IV

TABLE OF SEQUENCES

| SEQ ID | | |
|---|---|---|
| NO | Description | Sequence |
| 1 | LKB1 | atggaggtgg tggacccgca gcagctgggc atgttcacgg agggcgagct |
| | coding | gatgtcggtg ggtatggaca cgttcatcca ccgcatcgac tccaccgagg |
| | sequence | tcatctacca gccgcgccgc aagcgggcca agctcatcgg caagtacctg |
| | | atgggggacc tgctggggga aggctcttac ggcaaggtga aggaggtgct |
| | | ggactcggag acgctgtgca ggagggccgt caagatcctc aagaagaaga |
| | | agttgcgaag gatccccaac ggggaggcca acgtgaagaa ggaaattcaa |
| | | ctactgagga ggttacggca caaaaatgtc atccagctgg tggatgtgtt |
| | | atacaacgaa gagaagcaga aaatgtatat ggtgatggag tactgcgtgt |
| | | gtggcatgca ggaaatgctg gacagcgtgc cggagaagcg tttcccagtg |
| | | tgccaggccc acgggtactt ctgtcagctg attgacggcc tggagtacct |
| | | gcatagccag ggcattgtgc acaaggacat caagccgggg aacctgctgc |
| | | tcaccaccgg tggcaccctc aaaatctccg acctgggcgt ggccgaggca |
| | | ctgcacccgt tcgcggcgga cgacacctgc cggaccagcc agggctcccc |
| | | ggctttccag ccgcccgaga ttgccaacgg cctggacacc ttctccggct |
| | | tcaaggtgga catctggtcg gctggggtca ccctctacaa catcaccacg |
| | | ggtctgtacc ccttcgaagg ggacaacatc tacaagttgt ttgagaacat |
| | | cgggaagggg agctacgcca tcccgggcga ctgtggcccc ccgctctctg |
| | | acctgctgaa agggatgctt gagtacgaac cggccaagag gttctccatc |
| | | cggcagatcc ggcagcacag ctggttccgg aagaaacatc ctccggctga |
| | | agcaccagtg cccatcccac cgagcccaga caccaaggac cggtggcgca |
| | | gcatgactgt ggtgccgtac ttggaggacc tgcacggcgc ggacgaggac |
| | | gaggacctct tcgacatcga ggatgacatc atctacactc aggacttcac |
| | | ggtgcccgga caggtcccag aagaggaggc cagtcacaat ggacagcgcc |
| | | ggggcctccc caaggccgtg tgtatgaacg gcacagaggc ggcgcagctg |
| | | agcaccaaat ccagggcgga gggccgggcc cccaaccctg cccgcaaggc |
| | | ctgctccgcc agcagcaaga tccgccggct gtcggcctgc aagcagcagt |
| | | ga |
| 2 | LKB1 | MEVVDPQQLG MFTEGELMSV GMDTFIHRID STEVIYQPRR KRAKLIGKYL |
| | amino | MGDLLGEGSY GKVKEVLDSE TLCRRAVKIL KKKKLRRIPN GEANVKKEIQ |
| | acid | LLRRLRHKNV IQLVDVLYNE EKQKMYMVME YCVCGMQEML DSVPEKRFPV |
| | sequence | CQAHGYFCQL IDGLEYLHSQ GIVHKDIKPG NLLLTTGGTL KISDLGVAEA |
| | | LHPFAADDTC RTSQGSPAFQ PPEIANGLDT FSGFKVDIWS AGVTLYNITT |
| | | GLYPFEGDNI YKLFENIGKG SYAIPGDCGP PLSDLLKGML EYEPAKRFSI |
| | | RQIRQHSWFR KKHPPAEAPV PIPPSPDTKD RWRSMTVVPY LEDLHGADED |
| | | EDLFDIEDDI IYTQDFTVPG QVPEEEASHN GQRRGLPKAV CMNGTEAAQL |
| | | STKSRAEGRA PNPARKACSA SSKIRRLSAC KQQ |

1. A method for predicting whether a cancer will respond to an EGFR inhibitor, comprising determining whether the cancer comprises a LKB1 mutation, wherein the presence of the LKB1 mutation indicates that the cancer will respond to the EGFR inhibitor.

2. A method of identifying a cancer patient who is likely to benefit from an EGFR inhibitor, comprising determining whether the patient's cancer comprises a LKB1 mutation, wherein the presence of the LKB1 mutation indicates that the cancer patient will likely benefit from the EGFR inhibitor.

3. A method of selecting a therapy for a cancer patient, comprising (a) determining whether the patient's cancer comprises a LKB1 mutation; and (b) if the patient's cancer comprises a LKB1 mutation, selecting an EGFR inhibitor for the therapy.

4. A method of treating a cancer in a mammal, comprising (a) determining whether the cancer comprises a LKB1 mutation; and (b) if the cancer comprises a LKB1 mutation, administering to the mammal a therapeutically effective amount of an EGFR inhibitor.

5. A method of treating a cancer comprising a LKB1 mutation in a mammal, comprising administering to the mammal having the cancer a therapeutically effective amount of an EGFR inhibitor.

6. The method of claim 5, wherein prior to administering the EGFR inhibitor, the cancer was determined to comprise a LKB1 mutation.

7. The method of claim 4, wherein the cancer is a solid tumor.

8. The method of claim 7, wherein the cancer is selected from a large cell carcinoma, carcinoid cancer, cancer of neuroendrocrine origin, head and neck squamous cell carcinoma (HNSCC), colorectal cancer, cervical cancer, melanoma, skin cancer, leiomyoma, gastric cancer, glioblastoma, ovarian cancer, small cell lung cancer (SCLC), non-small cell lung cancer (NSCLC), pancreatic cancer, esophageal cancer, gastric cancer and thyroid cancer.

9. The method of claim 7, wherein the cancer is selected from lung cancer, pancreatic cancer, colorectal cancer, and head and neck cancer.

10. The method of claim 4, wherein the cancer is in a tissue selected from the tissues in Table 2.

11. The method of claim 4, wherein the LKB1 mutation comprises a variation in a LKB1 polynucleotide.

12. The method of claim 11, wherein the variation in the LKB1 polynucleotide is in the coding sequence of a LKB1 polynucleotide.

13. The method of claim 12, wherein the variation in the LKB1 polynucleotide comprises at least one variation selected from an insertion, a deletion, an inversion, and a substitution.

14. The method of claim 12, wherein the variation in the LKB1 polynucleotide results in a frame shift in the LKB1 coding sequence.

15. The method of claim 12, wherein the variation in the LKB1 polynucleotide results in a variation in the LKB1 polypeptide.

16. The method of claim 15, wherein the variation in the LKB1 polypeptide is selected from an insertion, a substitution, a deletion, and a truncation.

17. The method of claim 15, wherein at least one variation in the LKB1 polypeptide is an amino acid variation at an amino acid position selected from Table 1 that results in (a) significantly reduced or absent levels of LKB1 protein and/or (b) expression of a LKB1 protein with significantly reduced activity.

18. The method of claim 17, wherein at least one variation in the LKB1 polypeptide is an amino acid change selected from Table 1.

19. The method of claim 11, wherein the variation in the LKB1 polynucleotide is a nucleotide variation at a nucleotide position selected from Table 1 that results in (a) significantly reduced or absent levels of LKB1 protein and/or (b) expression of a LKB1 protein with significantly reduced activity.

20. The method of claim 19, wherein the variation in the LKB1 polynucleotide is a nucleotide change selected from Table 1.

21. The method of claim 4, wherein the mammal is a human.

22. The method of claim 4, wherein the EGFR inhibitor is an antibody that binds EGFR.

23. The method of claim 22, wherein the EGFR inhibitor is cetuximab or panitumumab.

24. The method of claim 4, wherein the EGFR inhibitor is a small molecule.

25. The method of claim 24, wherein the EGFR inhibitor is erlotinib or gefitinib.
